(19) United States
(12) Patent Application Publication
(10) Pub. No.: US 2018/0125600 A1
(43) Pub. Date: May 10, 2018

(54) Sizer Instrument And Methods Thereof

(71) Applicant: In2Bones USA, LLC, Memphis, TN (US)

(72) Inventors: Rebecca Hawkins Wahl, Escondido, CA (US); Casey Chambers, Memphis, TN (US); Jim Miller, Memphis, TN (US)

(73) Assignee: In2Bones USA, LLC, Memphis, TN (US)

(21) Appl. No.: 15/796,437

(22) Filed: Oct. 27, 2017

Related U.S. Application Data
(60) Provisional application No. 62/418,710, filed on Nov. 7, 2016.

(51) Int. Cl. A61B 90/00; A61B 17/80; A61B 17/17

(52) U.S. Cl. CPC A61B 90/06; A61B 17/0642; A61B 17/1728; A61B 17/80

(57) ABSTRACT

Provided herein in some embodiments is an instrument including an annular body, a plurality of caliper-tip pairs, and a plurality of through holes. The annular body includes an inner perimeter portion, an outer perimeter portion, and a medial portion in-between the inner perimeter portion and the outer perimeter portion. The plurality of caliper-tip pairs are disposed about the outer perimeter portion of the annular body, wherein each caliper-tip pair of the plurality of caliper-tip pairs has a different fixed caliper-tip spacing. The plurality of through holes are disposed about the medial portion of the annular body in pairs, wherein each pair of through holes of the plurality of through holes has a through-hole spacing matching the fixed caliper-tip spacing of at least one caliper-tip pair of the plurality of caliper-tip pairs.

### PRIORITY

This application claims the benefit of and priority to U.S. Provisional Application, entitled “Sizer Instrument And Methods Thereof,” filed on Nov. 7, 2016 and having application Ser. No. 62/418,710.

### FIELD

The field of the present disclosure generally relates to securing bones together. More particularly, the field of the present disclosure relates to a sizer instrument that a surgeon may utilize to size surgical staples during a medical procedure.

### BACKGROUND

Surgical staples or a fusion bone plate implant utilized in conjunction with one or more fasteners may be used to generate compression and stability at a bone interface. An implant generally serves to stabilize bones, or bone parts, relative to one another so as to promote bone fusion. In many applications, surgical staples, or bone plates and fasteners are used to fuse bones, or bone parts, of the human body, such as bones in the foot, the ankle, the hand, the wrist, as well as various other portions of the body. Surgical staples are particularly advantageous in the hands and feet due to a low dorsal profile of the staples once they are implanted. Furthermore, during the course of certain medical procedures, a surgeon may immobilize one or more bones or bone fragments by stabilizing the bones together in a configuration which approximates the natural anatomy. To this end, the surgeon may use fasteners to attach the bones to a bone plate implant so as to hold the bones in alignment with one another while they fuse together.

### SUMMARY

Provided herein in some embodiments is an instrument including an annular body, a plurality of caliper-tip pairs, and a plurality of through holes. The annular body includes an inner perimeter portion, an outer perimeter portion, and a medial portion in-between the inner perimeter portion and the outer perimeter portion. The plurality of caliper-tip pairs are disposed about the outer perimeter portion of the annular body, wherein each caliper-tip pair of the plurality of caliper-Application tip pairs has a different fixed caliper-tip spacing. The plurality of through holes are disposed about the medial portion of the annular body in pairs, wherein each pair of through holes of the plurality of through holes has a through-hole spacing matching the fixed caliper-tip spacing of at least one caliper-tip pair of the plurality of caliper-tip pairs.

In an exemplary embodiment, an instrument comprises an annular body including an inner perimeter portion, an outer perimeter portion, and a medial portion between the inner perimeter portion and the outer perimeter portion; a plurality of caliper-tip pairs disposed about the outer perimeter portion of the annular body, each of the plurality of caliper-tip pairs having a different fixed caliper-tip spacing; and a plurality of through holes disposed about the medial portion of the annular body in pairs, each of the plurality of through holes having a spacing that matches the fixed caliper-tip spacing of at least one of the plurality of caliper-tip pairs.

In another exemplary embodiment, the annular body is physically sectioned into a plurality of physical sections, and wherein each of the plurality of physical sections includes a space-matching pair of caliper tips and through holes. In another exemplary embodiment, the plurality of physical sections is comprised of seven physical sections. In another exemplary embodiment, the annular body is cupped, such that the inner perimeter portion and the outer perimeter portion of the annular body are situated in different planes. In another exemplary embodiment, the instrument further comprises a size indicator embossed, engraved, or printed in each of the plurality of physical sections about the medial portion of the annular body, such that each size indicator corresponds to a specific size of a surgical staple. In another exemplary embodiment, the instrument further comprises a plurality of caliper tip-connecting members, wherein each of the plurality of caliper tip-connecting members connects the caliper tips comprising at least one of the plurality of caliper-tip pairs to form a flange.

In another exemplary embodiment, the instrument is characteristic of being formed in one or more stages of a stamping process. In another exemplary embodiment, the instrument is stamped from stainless steel, an alloy of titanium and aluminum, or a plastic with sufficient hardness to withstand the one or more stages of the stamping process. In another exemplary embodiment, the instrument is characteristic of being formed in one or more stages of a molding process. In another exemplary embodiment, the instrument is comprised of polycarbonate, polyether ether ketone (“PEEK”), or polyetherimide.

In an exemplary embodiment, a staple sizer for determining sizes of surgical staples during surgery comprises a disc-shaped member comprising a plurality of sections corresponding to different sizes of surgical staples; a pair of parallel caliper-tips disposed within each of the plurality of sections and extending away from the center of the disc-shaped member; and a pair of holes disposed adjacently to each pair of parallel caliper-tips.

In another exemplary embodiment, each of the plurality of sections comprises a size indicator configured to display a size of the surgical stable in a distracted state, such that the parallel caliper-tips match the positions of a first leg and a second leg of the surgical staple. In another exemplary embodiment, the parallel caliper-tips are configured to correspond to the positions of the first leg and the second leg of specific size of the surgical staple. In another exemplary embodiment, the pair of holes are configured to indicate a relationship between holes in bone suitably drilled across a bone fusion or fixation site of a patient, the pair of holes corresponding to positions of a first leg and a second leg of the surgical staple in a distracted state.

In another exemplary embodiment, the plurality of sections includes seven sections. In another exemplary embodiment, the disc-shaped member is cupped, such that an inner perimeter portion and an outer perimeter portion of the disc-shaped member are situated in different planes. In another exemplary embodiment, a size indicator is embossed, engraved, or printed in each of the plurality of sections about a medial portion of the disc-shaped member, and wherein each size indicator corresponds to a specific size of the surgical staple.

In another exemplary embodiment, the staple sizer is characteristic of being formed in one or more stages of a stamping process. In another exemplary embodiment, the staple sizer is stamped from stainless steel, an alloy of titanium and aluminum, or a plastic with sufficient hardness to endure the one or more stages of the stamping process. In another exemplary embodiment, the staple sizer is characteristic of being formed in one or more stages of a molding process. In another exemplary embodiment, the staple sizer is comprised of polycarbonate, polyether ether ketone (“PEEK”), or polyetherimide.

These and other features of the concepts provided herein may be better understood with reference to the drawings, description, and appended claims.

### BRIEF DESCRIPTION OF THE DRAWINGS

The drawings refer to embodiments of the present disclosure in which:

FIG. 1 illustrates a top plan view of an exemplary embodiment of a sizer instrument in accordance with the present disclosure;

FIG. 2 illustrates a side plan view of an exemplary embodiment of a sizer instrument, according to the present disclosure;

FIG. 3 illustrates an upper perspective view of an exemplary embodiment of a sizer instrument, according to the present disclosure; and

FIG. 4 illustrates a lower perspective view of an exemplary embodiment of a sizer instrument in accordance with the present disclosure.

While the present disclosure is subject to various modifications and alternative forms, specific embodiments thereof have been shown by way of example in the drawings and will herein be described in detail. The invention should be understood to not be limited to the particular forms disclosed, but on the contrary, the intention is to cover all modifications, equivalents, and alternatives falling within the spirit and scope of the present disclosure.

### DETAILED DESCRIPTION

In the following description, numerous specific details are set forth in order to provide a thorough understanding of the present disclosure. It will be apparent, however, to one of ordinary skill in the art that the invention disclosed herein may be practiced without these specific details. In other instances, specific numeric references such as “first implant,” may be made. However, the specific numeric reference should not be interpreted as a literal sequential order but rather interpreted that the “first implant” is different than a “second implant.” Thus, the specific details set forth are merely exemplary. The specific details may be varied from and still be contemplated to be within the spirit and scope of the present disclosure. The term “coupled” is defined as meaning connected either directly to the component or indirectly to the component through another component. Further, as used herein, the terms “about,” “approximately,” or “substantially” for any numerical values or ranges indicate a suitable dimensional tolerance that allows the part or collection of components to function for its intended purpose as described herein.

Moreover, it should also be understood the terminology used herein is intended for the purpose of describing some particular embodiments, and the terminology does not limit the scope of the concepts provided herein. Unless indicated otherwise, ordinal numbers (e.g., first, second, third, etc.) are used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. It should also be understood that, unless indicated otherwise, any labels such as “left,” “right,” “front,” “back,” “top,” “bottom,” “forward,” “reverse,” “clockwise,” “counter clockwise,” “up,” “down,” or other similar terms such as “upper,” “lower,” “aft,” “fore,” “vertical,” “horizontal,” “proximal,” “distal,” and the like are used for convenience and are not intended to imply, for example, any particular fixed location, orientation, or direction. Instead, such labels are used to reflect, for example, relative location, orientation, or directions. It should also be understood that the singular forms of “a,” “an,” and “the” include plural references unless the context clearly dictates otherwise.

Unless defined otherwise, all technical and scientific terms used herein have the same meaning as commonly understood those of ordinary skill in the art.

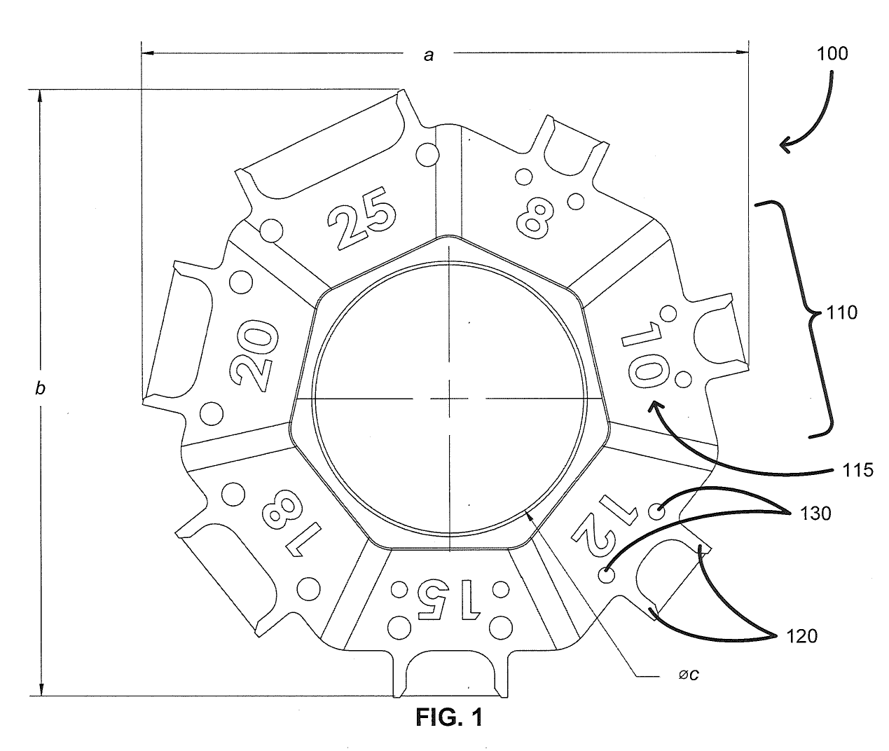

FIG. 1 illustrates a top plan view of an exemplary embodiment of a sizer instrument 100 in accordance with the present disclosure. As shown in FIG. 1, the sizer instrument 100 can have an annular form. It should be appreciated that the sizer instrument 100 need not be limited to such annular forms. For example, linear forms can also be used.

The sizer instrument 100 can have a number of sections such as section 110, each of which can be generally trapezoidal in shape, and each of which corresponds to a different size of surgical staple and drill holes drilled therefor. In some embodiments, for example, the number of sections of the sizer instrument 100 can include three, four, five, six, seven, eight, nine, ten, or more than ten sections. In linear forms, the number of sections can contribute to an overall length of the sizer instrument 100. In annular forms, the number of sections can correspond to a number of sides of the sizer instrument 100, and, thus, contribute to a shape of the sizer instrument 100. Three sections provide a triangle-shaped sizer instrument, four sections provide a square-shaped sizer instrument, five sections provide a pentagon-shaped sizer instrument, six sections provide a hexagon-shaped sizer instrument, seven sections provide a heptagon-shaped sizer instrument, and so on. As shown in FIG. 1, the sizer instrument 100 can have seven sections providing a heptagonal shape to the sizer instrument 100. It has been found that the seven sections of the sizer instrument 100 are useful for a number of operating-room procedures, and the heptagon shape results in dimensions for a hand-held instrument suitable for most surgeons.

Dimensions can vary with the number of sections of the sizer instrument 100. As shown in FIG. 1, the sizer instrument 100 can have a length a and a width b, which length a and width b can be the same or different. For annular forms of the sizer instrument 100 such as the heptagonal-shaped sizer instrument of FIG. 1, a and b can be the same. In some embodiments, for example, the length a and the width b can be at least 1.5″, 3″, 3.25″, 3,5″, or 4.5″, including fractions (e.g., tenths or hundredths) thereof. Also for annular forms of the sizer instrument 100 such as the heptagonal-shaped sizer instrument of FIG. 1, the sizer instrument 100 can have an inner diameter c. In some embodiments, for example, the diameter c can be at least 1″, 1.25″, 1.5″, 1.75″, or 2″, including fractions (e.g., tenths or hundredths) thereof. Again, it has been found that the sizer instrument 100 with such dimensions provides a hand-held instrument suitable for most surgeons.

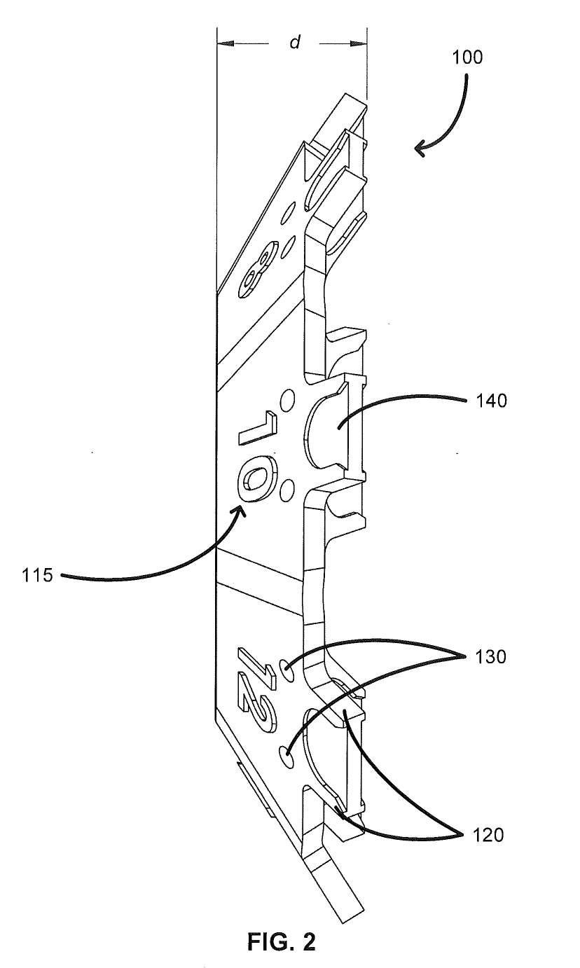

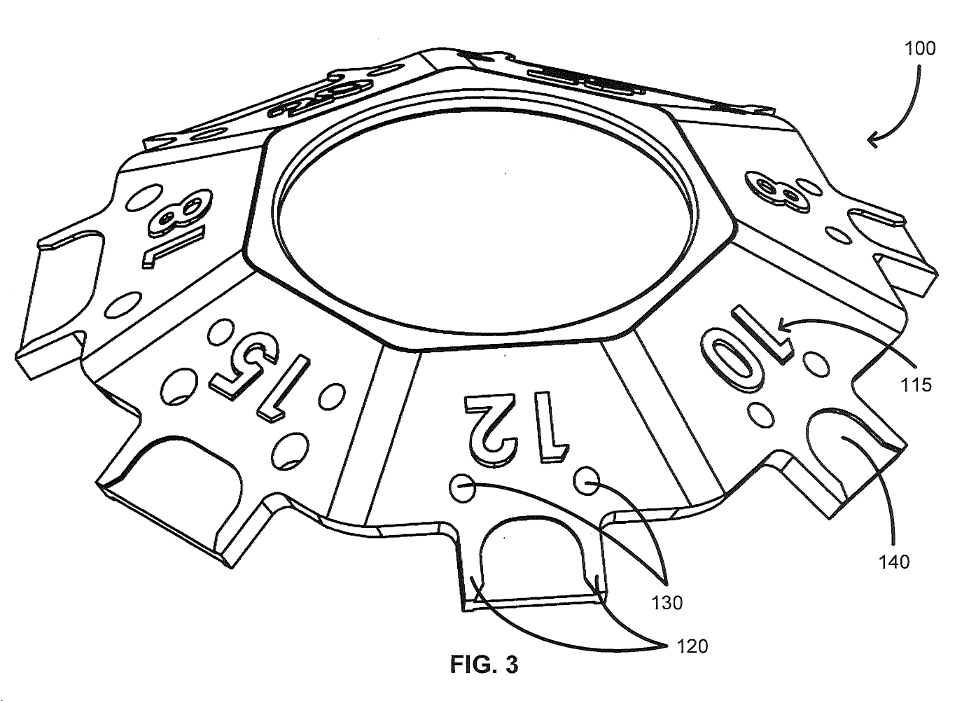

As shown in FIG. 1, each section of the number of sections can include a pair of caliper tips or caliper tip-like protrusions 120 extending radially outward from the sizer instrument 100 and a medial section including at least one pair of through holes 130 passing through the medial section and a number or size indicator 115 (e.g., a number of one or more Arabic numerals) embossed (see FIGS. 2 and 3), engraved, printed, or the like in the medial section. The pair of protrusions 120 can have a first, fixed spacing between the protrusions, and the pair of through holes 130 can have a second spacing between the through holes, wherein the first spacing and the second spacing are the same. The medial-section number for each section of the number of sections indicates both the first spacing and the second spacing. For example, the section 110 of FIG. 1 having a medial section labeled “10” indicates the first spacing between the pair of protrusions and the second spacing between the pair of through holes is 10 mm. Adjacent to the section 110 of FIG. 1 is a section labeled “12” indicating the first spacing between the pair of protrusions and the second spacing between the pair of through holes is 12 mm. Continuing in a clockwise fashion, the sizer instrument 100 of FIG. 1 also includes sections for 15 mm, 18 mm, 20 mm, 25 mm, and 8 mm spacings between corresponding pairs of protrusions and pairs of through holes.

Again, each section of the number of sections can correspond to a different size of surgical staple and drill holes drilled therefor. While the medial-section number for each section of the number of sections indicates both the first spacing between the protrusions and the second spacing between the through holes, it can also correspond a third spacing between legs of a surgical staple. For example, while the section 110 of FIG. 1 having a medial section labeled “10” indicates the first spacing between the pair of protrusions and the second spacing between the pair of through holes is 10 mm, the section 110 also corresponds to a third spacing of 10 mm between legs of a surgical staple. In view of the foregoing, a surgeon can visually align a pair of caliper tips of caliper tip-like protrusions with a pair of drill holes (e.g., drill holes suitably drilled across a bone fusion or fixation site of a patient) to select—by the corresponding medial-section number—a surgical staple with an appropriate leg spacing for inserting into the drill holes. Using the pair of through holes corresponding to the medial-section number, the surgeon can subsequently check the spacing between the legs of the selected surgical staple by inserting the legs of the surgical staple into the pair of through holes prior to inserting the legs of the surgical staple into the drill holes.

FIG. 2 illustrates a side plan view of the sizer instrument 100, according to the present disclosure.

The sizer instrument 100 can have an annular form as provided in reference to FIG. 1. In addition, the sizer instrument 100 can be cupped such that the inner diameter c and an outer diameter defined by the pairs of protrusions 120 lie in different planes separated by a height or distance d. In some embodiments, for example, the distance d can be at least 0.25″, 0.5″, or 0.75″, including fractions (e.g., tenths or hundredths) thereof. It should be appreciated that the sizer instrument 100 need not be cupped. However, it has been found that the sizer instrument 100 cupped with such dimensions provides an ergonomic hand-held instrument suitable for most surgeons to comfortably hold between their thumb and forefinger without overpronation.

As shown in FIG. 2, each pair of protrusions 120 in each section of the number of sections can include a caliper tip-connecting or protrusion-connecting member 140 between the pair of protrusions, wherein the protrusion-connecting member 140 extends radially outward from the sizer instrument 100 forming a flange with the pair of protrusions it connects. The protrusion-connecting member 140 can extend radially outward from the sizer instrument 100 as far as the pair of protrusions it connects, or the protrusion-connecting member 140 can extend radially outward from the sizer instrument 100 a shorter distance than the pair of protrusions it connects. In some embodiments, for example, the shorter distance can be no more than 0.125″, 0.09375″, 0.0625″, or 0.03125,″ including fractions thereof. It should be appreciated that the sizer instrument 100 need not include protrusion-connecting members. However, it has been found that the protrusion-connecting members can minimize or eliminate any risks of wounding the patient and operating-room personnel with sizer instrument 100 and a pairs of protrusion thereof while maintaining utility of the sizer utility instrument 100.

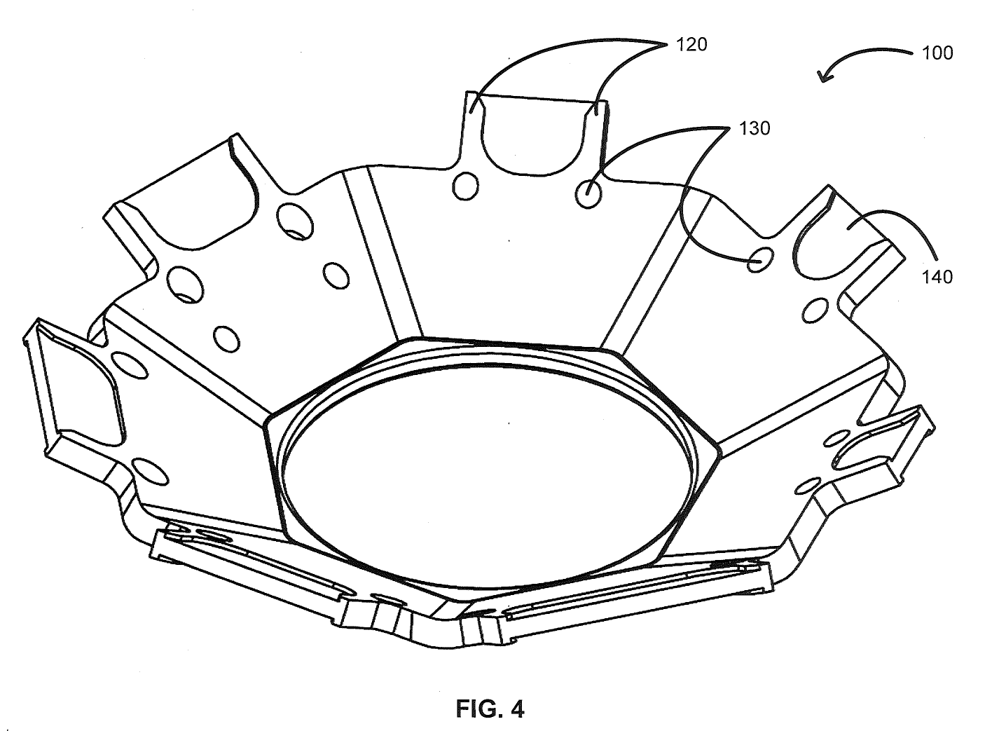

FIGS. 3 and 4 respectively provide an upper perspective view and a lower perspective view of the sizer instrument 100 in accordance with the present disclosure. FIGS. 3 and 4 use the same reference numbers as FIGS. 1 and 2 to refer to the same structural elements.

The sizer instrument 100 can be made by any process and of any surgical grade material that can be sufficiently sterilized for use in an operating room for a medical procedure (e.g., osteotomy procedures). In some embodiments, for example, the sizer instrument 100 can be stamped from a surgical grade material in a one-stage or a multiple-stage stamping process. It will be appreciated that the stamping process can be used to create embossed numbers such as the embossed number 115 shown in FIGS. 2 and 3. The surgical grade material for the stamping process can include, but is not limited to, stainless steel; alloys of titanium and aluminum including titanium-aluminum-vanadium alloys and titanium-aluminum-niobium alloys; or certain plastics with sufficient hardness without being to brittle. In some embodiments, for example, the sizer instrument 100 can be molded from a surgical grade material in a one-stage or a multiple-stage molding process. It will be appreciated that the molding process can also be used to create embossed numbers such as the embossed number 115 shown in FIGS. 2 and 3. The surgical grade material for the molding process (e.g., injection molding) can include, but is not limited to, polycarbonate, polyether ether ketone (“PEEK”), or polyetherimide.

As provided above, it is envisioned that the sizer instrument 100 can be utilized during a medical procedure to assist a surgeon in selecting an appropriately sized surgical staple. For example, after drilling osteotomy holes across a bone fusion or fixation site of a patient, the surgeon can compare the drill holes to one or more pairs of protrusions 120 of the sizer instrument 100. Upon finding a matching pair of protrusions that best matches the drill holes, the surgeon can—using the corresponding size indicator embossed, engraved, or printed in the medial section corresponding to the matching pair of protrusions—determine and select the appropriately sized surgical staple for implantation into the drill holes of the osteotomy patient. The surgeon can subsequently compare the spacing between the legs of the selected staple with the matching pair of through holes per the size indicator to verify the suitability of the selected surgical staple for implantation into the drill holes of the osteotomy patient. As will be appreciated, the sizer instrument 100 effectively enables a surgeon to select an appropriately sized surgical staple and confirm the appropriateness of the selected surgical staple.

As such, provided herein in some embodiments is an instrument including an annular body, a plurality of caliper-tip pairs, and a plurality of through holes. The annular body includes an inner perimeter portion, an outer perimeter portion, and a medial portion in-between the inner perimeter portion and the outer perimeter portion. The plurality of caliper-tip pairs are disposed about the outer perimeter portion of the annular body, wherein each caliper-tip pair of the plurality of caliper-tip pairs has a different fixed caliper-tip spacing. The plurality of through holes are disposed about the medial portion of the annular body in pairs, wherein each pair of through holes of the plurality of through holes has a through-hole spacing matching the fixed caliper-tip spacing of at least one caliper-tip pair of the plurality of caliper-tip pairs. In some embodiments, the annular body is physically sectioned into a plurality of physical sections, and each physical section of the plurality of physical sections includes a space-matching pair of caliper tips and through holes. In some embodiments, the plurality of physical sections includes seven physical sections. In some embodiments, the annular body is cupped, and the inner perimeter portion and the outer perimeter portion of the annular body lie in different planes. In some embodiments, the instrument further comprises a size indicator embossed, engraved, or printed in each physical section about the medial portion of the annular body, wherein each size indicator of the plurality of size indicators corresponds to a surgical staple of a different size. In some embodiments, the instrument further comprises a plurality of caliper tip-connecting members, wherein each caliper tip-connecting member of the plurality of caliper tip-connecting members connects the caliper tips of at least one caliper-tip pair of the plurality of caliper-tip pairs to form a flange. In some embodiments, the instrument is characteristic of being formed in one or more stages of a stamping process. In some embodiments, the instrument is stamped from stainless steel; an alloy of titanium and aluminum; or a plastic with sufficient hardness without being to brittle for the one or more stages of the stamping process. In some embodiments, the instrument is characteristic of being formed in one or more stages of a molding process. In some embodiments, the instrument is molded from polycarbonate, polyether ether ketone (“PEEK”), or polyetherimide.

While the invention has been described in terms of particular variations and illustrative figures, those of ordinary skill in the art will recognize that the invention is not limited to the variations or figures described. In addition, where methods and steps described above indicate certain events occurring in certain order, those of ordinary skill in the art will recognize that the ordering of certain steps may be modified and that such modifications are in accordance with the variations of the invention. Additionally, certain of the steps may be performed concurrently in a parallel process when possible, as well as performed sequentially as described above. To the extent there are variations of the invention, which are within the spirit of the disclosure or equivalent to the inventions found in the claims, it is the intent that this patent will cover those variations as well. Therefore, the present disclosure is to be understood as not limited by the specific embodiments described herein, but only by scope of the appended claims.

What is claimed is:

1. An instrument, comprising:
an annular body including an inner perimeter portion, an outer perimeter portion, and a medial portion between the inner perimeter portion and the outer perimeter portion;
a plurality of caliper-tip pairs disposed about the outer perimeter portion of the annular body, each of the plurality of caliper-tip pairs having a different fixed caliper-tip spacing; and
a plurality of through holes disposed about the medial portion of the annular body in pairs, each of the plurality of through holes having a spacing that matches the fixed caliper-tip spacing of at least one of the plurality of caliper-tip pairs.

2. The instrument of claim 1, wherein the annular body is physically sectioned into a plurality of physical sections, and wherein each of the plurality of physical sections includes a space-matching pair of caliper tips and through holes.

3. The instrument of claim 2, wherein the plurality of physical sections is comprised of seven physical sections.

4. The instrument of claim 2, wherein the annular body is cupped, such that the inner perimeter portion and the outer perimeter portion of the annular body are situated in different planes.

5. The instrument of claim 2, further comprising a size indicator embossed, engraved, or printed in each of the plurality of physical sections about the medial portion of the annular body, such that each size indicator corresponds to a specific size of a surgical staple.

6. The instrument of claim 1, further comprising a plurality of caliper tip-connecting members, wherein each of the plurality of caliper tip-connecting members connects the caliper tips comprising at least one of the plurality of caliper-tip pairs to form a flange.

7. The instrument of claim 1, wherein the instrument is characteristic of being formed in one or more stages of a stamping process.

8. The instrument of claim 7, wherein the instrument is stamped from stainless steel, an alloy of titanium and aluminum, or a plastic with sufficient hardness to withstand the one or more stages of the stamping process.

9. The instrument of claim 1, wherein the instrument is characteristic of being formed in one or more stages of a molding process.

10. The instrument of claim 9, wherein the instrument is comprised of polycarbonate, polyether ether ketone (“PEEK”), or polyetherimide.

11. A staple sizer for determining sizes of surgical staples during surgery, comprising:
a disc-shaped member comprising a plurality of sections corresponding to different sizes of surgical staples;
a pair of parallel caliper-tips disposed within each of the plurality of sections and extending away from the center of the disc-shaped member; and
a pair of holes disposed adjacently to each pair of parallel caliper-tips.

12. The staple sizer of claim 11, wherein each of the plurality of sections comprises a size indicator configured to display a size of the surgical stable in a distracted state, such that the parallel caliper-tips match the positions of a first leg and a second leg of the surgical staple.

13. The staple sizer of claim 13, wherein the parallel caliper-tips are configured to correspond to the positions of the first leg and the second leg of specific size of the surgical staple.

14. The staple sizer of claim 11, wherein the pair of holes are configured to indicate a relationship between holes in bone suitably drilled across a bone fusion or fixation site of a patient, the pair of holes corresponding to positions of a first leg and a second leg of the surgical staple in a distracted state.

15. The staple sizer of claim 11, wherein the plurality of sections includes seven sections.

16. The staple sizer of claim 11, wherein the disc-shaped member is cupped, such that an inner perimeter portion and an outer perimeter portion of the disc-shaped member are situated in different planes.

17. The staple sizer of claim 11, wherein a size indicator is embossed, engraved, or printed in each of the plurality of sections about a medial portion of the disc-shaped member, and wherein each size indicator corresponds to a specific size of the surgical staple.

18. The staple sizer of claim 11, wherein the staple sizer is characteristic of being formed in one or more stages of a stamping process.

19. The staple sizer of claim 18, wherein the staple sizer is stamped from stainless steel, an alloy of titanium and aluminum, or a plastic with sufficient hardness to endure the one or more stages of the stamping process.

20. The staple sizer of claim 11, wherein the staple sizer is characteristic of being formed in one or more stages of a molding process.

21. The staple sizer of claim 20, wherein the staple sizer is comprised of polycarbonate, polyether ether ketone (“PEEK”), or polyetherimide.
